(19) United States
(12) Patent Application Publication
(10) Pub. No.: US 2004/0033963 A1
(43) Pub. Date: Feb. 19, 2004

(54) Urea composition

(76) Inventors: Ruey J. Yu, Ambler, PA (US); Eugene J. Van Scott, Abington, PA (US)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/409,684

(22) Filed: Apr. 9, 2003

Related U.S. Application Data
(60) Provisional application No. 60/371,157, filed on Apr. 10, 2002.

(51) Int. Cl. A61K 31/70; A61K 31/366; A61K 31/19

(52) U.S. Cl. 514/23; 514/460; 514/557; 514/574

(57) ABSTRACT

The invention is directed to compositions, methods of making the compositions, and methods of treating cosmetic and dermatological disorders with a composition that includes a molecular complex between urea and a functional substance that has at least one hydroxyl group and one carboxyl group either as a free acid, a salt, an amide or a lactone. The compositions are stable when compared to conventional urea-containing compositions, and provide controlled-release of the urea.

### BACKGROUND OF THE INVENTION

1. Field of the Invention

This application relates to urea compositions including a molecular complex formed between at least one urea molecule and a functional substance. The composition containing urea in molecular complex is chemically stable and is therapeutically effective under control-release for topical treatment of various cosmetic indications and dermatological disorders.

2. Description of Related Art

Urea is a white crystalline organic compound having the chemical formula, H₂NCONH₂, a molecular weight of 60, and a melting point 133° C. According to “Textbook of Organic Medicinal and Pharmaceutical Chemistry,” Wilson, C. O., Gisvold, O., and Doerge, R. F., Editors, J. B. Lippincott Company, 6th Edition, (1971), page 190, urea has some antiseptic action and has been used for topical treatment of infected wounds by promoting granulation and healing, as well as removing dead tissues. Urea solutions also have been used to treat warts by injection. According to “Textbook of Dermatology,” Champion, R. H., Burton, J. L., and Ebling, F. J. G., Editors, Blackwell Scientific Publications, 5th Edition, (1992), page 3052, urea can accelerate the digestion of fibrin at 15%, and it is proteolytic at 40% strength by solubilizing and denaturing proteins. Urea at a concentration of about 10% in oil-in-water cream also has been used for topical treatment of ichthyosis and dry skin conditions. Urea as a 40% aqueous solution has been used for treatment of black hairy tongue and for acne conglobata. “Current Dermatological Management,” Maddin, S., and Brown, T. H., Editors, The C. V. Mosby Company, (1975), page 196, discloses the use of urea as a 10 to 40% cream for topical treatment of follicular keratoses, such as keratosis pilaris, keratosis spinulosa and keratosis pilaris atrophicans. “Basic & Clinical Pharmacology,” Katzung, B. G., Editor, Appleton & Lange, (1995), page 944, discloses the use of urea at concentrations of about 2 to 20% in creams and lotions as a humectant. Urea at 20% concentration also has been used as a keratolytic agent for topical treatment of ichthyosis vulgaris, hyperkeratosis of palms and soles, xerosis and keratosis pilaris. Finally, urea at concentrations of about 30 to 50% in ointments has been applied to nail plate under occlusion to soften the nail.

U.S. Pat. No. 3,666,863, entitled “Skin-Treating Composition and Vehicle for Skin-Treating Agents,” discloses and claims a topical composition comprising 2 to 30% urea and 0.5 to 8% lactic acid. Lactic acid is added to stabilize the urea because urea is known to decompose in aqueous formulation and produce ammonia. U.S. Pat. No. 5,919,470, entitled “Dermatological Composition,” discloses a topical composition comprising 21 to 40% urea in excipients. The excipients comprise skin protectants of an oleaginous nature derived from petrolatum, emulsifiers, and thickeners. U.S. Pat. No. 0.6,281,239, entitled “Method of Treating Onychomycosis,” discloses an antifungal composition including 40% urea and up to 5% antifungal agent for topical treatment of fungal infections. U.S. Pat. No. 6,380,236 entitled “Method of Treating Onychomycosis,” discloses an antifungal pack including an antifungal cream and a tissue softening cream containing urea for topical treatment of fungal infections. The disclosure of each of these patents is incorporated by reference herein in their entirety.

It is known that when urea is dissolved in water without a stabilizer, ammonia is produced slowly and the pH of the solution increases over the time. The instability of the urea formulation accelerates with the increased temperature, and urea decomposes to biuret, cyanuric acid and ammonia. The primary action of urea on the skin is keratolytic and the utility is limited to and only moderately effective for dry skin and as a humectant.

It also is known to form a molecular complex between an organic complexing compound and an alpha hydroxyacid or related acid for control release of the alpha hydroxyacid or related acid. U.S. Pat. No. 5,877,212, the disclosure of which is incorporated by reference herein in its entirety, discloses a molecular complex where the complexing agent has an amino group and at least one additional group that can form multiple hydrogen bonds with a free alpha hydroxyacid or related acid.

Numerous patents and publications by the present inventors describe and claim the use of a variety of alpha hydroxyacids and related acids for treatment of myriad dermatological disorders.

For example, in our prior U.S. Pat. No. 3,879,537 entitled “Treatment of Ichthyosiform Dermatoses,” we described and claimed the use of certain alpha hydroxyacids, alpha ketoacids and related compounds for topical treatment of fish-scale like ichthyotic conditions in humans. In our U.S. Pat. No. 3,920,835 entitled “Treatment of Disturbed Keratinization,” we described and claimed the use of these alpha hydroxyacids, alpha ketoacids and their derivatives for topical treatment of dandruff, acne, and palmar and plantar hyperkeratosis.

In our prior U.S. Pat. No. 4,105,783 entitled “Treatment of Dry Skin.” we described and claimed the use of non-irritating compositions containing reaction products formed between an alpha hydroxyacid or alpha ketoacid and ammonium hydroxide or an organic primary, secondary or tertiary alkyl amine or the like having from 1 to 8 carbon atoms, for topical treatment of dry skin. In our recent U.S. Pat. No. 4,246,261 entitled “Additives Enhancing Topical Corticosteroid Action,” we described and claimed that alpha hydroxyacids, alpha ketoacids and their derivatives could greatly enhance the therapeutic efficacy of corticosteroids in topical treatment of psoriasis, eczema, seborrheic dermatitis and other inflammatory skin conditions.

In our U.S. Pat. No. 4,363,815 entitled “Alpha Hydroxyacids, Alpha Ketoacids and Their Use in Treating Skin Conditions,” we described and claimed that alpha hydroxyacids and alpha ketoacids related to or originating from amino acids, whether or not found in proteins, were effective in topical treatment of skin disorders associated with disturbed keratinization or inflammation. These skin disorders include dry skin, ichthyosis, palmar and plantar hyperkeratosis, dandruff, Darier's disease, lichen simplex chronicus, keratoses, acne, psoriasis, eczema, pruritus, warts and herpes.

In our recent U.S. patent application Ser. No. 945,680 filed Dec. 23, 1986 now abandoned and entitled “Additives Enhancing Topical Actions of Therapeutic Agents,” we described among other things that incorporation of an alpha hydroxyacid or related compound can substantially enhance therapeutic actions of cosmetic and pharmaceutical agents. We also described methods of treating wrinkles and skin changes associated with aging using an alpha hydroxyacid or related compound.

In U.S. Pat. No. 5,091,171, entitled “Amphoteric Compositions and Polymeric Forms of Alpha Hydroxyacids, and Their Therapeutic Use,” we described among other things compositions containing an amphoteric complex formed between an alpha hydroxyacid or related compound and an amphoteric or pseudoamphoteric agent are therapeutically effective for topical treatment of various cosmetic conditions and dermatologic indications.

In U.S. Pat. No. 5,554,597 entitled “Compositions Comprising 2-Hydroxycarboxylic Acid and Related Compounds, and Methods for Alleviating Signs of Dermatologic Aging,” we described among other things that compositions containing an alpha hydroxyacid or related compound are therapeutically effective for topical treatment of dermatological signs of aging. The signs of aging include changes or damage to skin, nail and hair associated with intrinsic aging, as well as changes or damage caused by extrinsic factors such as sunlight, radiation, air pollution, wind, cold, heat, dampness, chemicals, smoke and cigarette smoking.

In recent U.S. Pat. No. 5,425,938 entitled “Polyamino Salts of Alpha-Hydroxyacids, Alpha-Ketoacids and Related Compounds,” it is disclosed that such polyamino salts might be used in cosmetic compositions. The claimed amino polymers have optimal molecular weights of from 10,000 to 800,000. However, according to Jackson S. M., Elias P. M.: SKIN AS AN ORGAN OF PROTECTION cited in Fitzpatrick T. B., Eisen A. Z., Wolff K., Freedberg I. M., Austen K. F. (ed.): DERMATOLOGY IN GENERAL MEDICINE, 4th edition, McGraw-Hill, Inc., New York; 1993: Chapter 16, 241-253, experiments have shown that even non-polar polymers with molecular weight of above 800-1000 decrease dramatically in penetration through the stratum corneum of the skin. Therefore, such amino polymers cannot readily penetrate the stratum corneum of human skin due to their high molecular weight and polar nature of the polyamino salt

Each of the foregoing patents and applications is expressly incorporated herein by reference in their entireties.

The description herein of certain disadvantages of known compositions and methods is not intended to limit the invention to exclude such known compositions and methods. Indeed, various aspects of the invention may include these known compositions and methods without suffering from their known disadvantages.

### SUMMARY OF THE INVENTION

It is a feature of an embodiment of the invention to provide a composition including a urea molecular complex that provides for controlled release of the urea. It also is a feature of an embodiment of the invention to provide a method of making the composition and a method of topical application of the composition to the skin for treatment of various cosmetic indications and dermatological disorders.

We have now discovered that urea can form a stable molecular complex with a functional substance, and that such complex is therapeutically effective for topical treatment of various cosmetic indications and dermatological disorders. The functional substance preferably is selected from the group consisting of hydroxyacids and polyhydroxy acids to provide for optimal bioavailability and controlled delivery of urea molecule into integumental tissues. The functional substances include glycolic acid, mandelic acid, benzilic acid, gluconic acid, gluconolactone, ribonolactone, galactonolactone, glucuronolactone and glucarolactone.

In accordance with an embodiment of the invention, the functional substance includes at least one hydroxyl group and one carboxyl group either as a free acid, amide or lactone form. Since the urea molecule consists of three functional groups, two amino and one carbonyl, the complex formation is based on dipolar/dipolar and dipolar/ionic attracting forces between the urea and the functional substance. The inventive composition containing molecular complex of urea is therapeutically effective for general care of skin, hair and nail; topical management and treatment of various cosmetic and dermatological indications including cosmetic and clinical signs of changes associated with intrinsic and extrinsic aging.

In accordance with these and other features of various embodiments of the invention, there is provided a composition comprising a molecular complex formed between urea and a functional substance comprising at least one hydroxyl group and one carboxyl group either as a free acid, salt, an amide or a lactone.

In accordance with another feature of an embodiment of the invention, there is provided a method of making a composition comprising a complex of urea and a functional substance comprising at least one hydroxyl group and one carboxyl group either as a free acid, an amide or a lactone, the method comprising forming a complex between urea and the functional substance, and formulating the complex into a topically acceptable vehicle.

In accordance with another feature of an embodiment of the invention, there is provided a method of treating a cosmetic or dermatological disorder comprising topically applying to an area of the skin containing the cosmetic or dermatological disorder, a therapeutically effective amount of a composition comprising a molecular complex formed between urea and a functional substance comprising at least one hydroxyl group and one carboxyl group either as a free acid, salt, an amide or a lactone.

### DETAILED DESCRIPTION OF THE PREFERRED EMBODIMENTS

Terms and phrases used herein are defined as set forth below unless otherwise specified. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are cited for the purpose of describing and disclosing the compounds, molecules, and methodologies that are reported in the publications and that might be used in connection with the invention. Nothing herein is to be construed as an admission that the invention is not entitled to antedate such disclosure by virtue of prior invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Throughout this description, the term “complex” and the phrase “molecular complex” denote a combination of two or more compounds, and is premised on dipolar/dipolar and dipolar/ionic intermolecular attracting forces between the urea and the functional substance.

When urea is dissolved in water to make 20 or 40% concentration, the pH is around 7.4. In the absence of forming a stabilized molecular complex, the aqueous urea solution will produce ammonia and the pH raises slowly. The generated ammonia and the raise of pH accelerates the self decomposition of urea molecules. For example, 20% and 40% urea solutions change from pH 7.4 to pH 8.8 and pH 9.0, respectively after 11 months at room temperature as shown in Table 1. In contrast, the stabilized urea compositions of the invention that contain a molecular complex with a functional group (e.g., hydroxyacid or polyhydroxy acid) are stable for an extended period of time.

TABLE 1
Urea Compositions

pH

| | Freshly Prepared | After 11 Months |
|---|---|---|
| Urea 20% | 7.4 | 8.8 |
| Urea 40% | 7.4 | 9.0 |
| Invention (20%) | 8.2^{a} | 8.0 |

^{a}Molecular complex with 5% glycolic acid and pH adjustment with L-arginine.

We have discovered that because the urea molecule has three functional groups, (two amino and one carbonyl), it can form a stabilized molecular complex with a functional substance based on intermolecular attracting forces. It is preferred that the functional substance include at least one hydroxyl group and one carboxyl group either as a free acid, salt, an amide or a lactone. More preferably, the functional substance is selected from the group consisting of hydroxyacids, polyhydroxy acids, and related acids. According to Organic Chemistry by T. W. Graham Solomons, 5^{th} edition, John Wiley & Sons, page 76-82, 1992, there are five attractive electric forces between molecular species. These forces include ionic/ionic, covalent bonds, dipolar/ionic, dipolar/dipolar (including hydrogen bonds) and van der Waals. While not intending on being bound by any theory of operation, we believe that only two major attractive forces, namely dipolar/dipolar and dipolar/ionic, operate between the urea molecule and the functional substance of the present invention. The dipolar/dipolar attractive force is believed to be created between the hydroxyl, amide or lactone group of the functional substance and the amino or carbonyl group of urea. The dipolar/ionic attractive force is believed to be created between the amino or carbonyl group of urea and the carboxyl group of the functional substance as shown in Table 2. The urea composition of the invention including a molecular complex has three major advantages, (a) stable composition, (b) control-release mechanism for urea molecule, and (c) therapeutically effective for wide range of cosmetic and dermatological indications.

TABLE 2
Attractive Electric Forces in Molecular Complex^{a}

| | | Functional Group |
|---|---|---|
| Attractive Force | Urea | (Hydroxyacid/Polyhydroxy Acid) |
| Dipolar/Dipolar | —NH | OH |
| | —C═O | HO |
| | —NH | O═CNH (in amide) |
| | —NH | O═C—O (in lactone) |
| Dipolar/Ionic | —NH | ⁻O—C═O |

^{a}Examples of involved radical groups.

In addition, some hydroxyacids and all polyhydroxy acids and lactones have been found to be antioxidants and can prevent air oxidation of urea composition. The molecular complex thus formed between urea and the functional substance is rather stable as shown by near constant or minimal pH change of the composition. The antioxidant property of some hydroxyacids, polyhydroxy acids and lactones are determined as follows. Oxidation by definition is removal of electrons or addition of oxygen. An antioxidant can be defined as a substance capable of preventing or inhibiting oxidation, or capable of disposing, scavenging, or suppressing formation or actions of peroxide, superoxide or free radicals. We have developed three simple assays to determine if a substance is an antioxidant or not. The antioxidant property is determined by using one of the following screening methods: prevention or retardation of air oxidation of (a) anthralin, (b) hydroquinone, or (c) banana peel. A freshly prepared anthralin solution or cream is bright yellow, and an air oxidized one is brownish or black. A hydroquinone solution or cream is colorless or white color, and an air-oxidized one is brownish or black. A freshly peeled banana peel is light yellow in color, and an oxidized one ranges in color from tan, dark tan, brown to brownish black.

Known antioxidants such as vitamin C and N-acetylcysteine may be used as the positive control in these screening methods. Based on these tests, the following hydroxyacids and polyhydroxy acids have been found to be antioxidants: citric acid, isocitric acid, tartaric acid, malic acid, tartronic acid, ascorbic acid, isoascorbic acid, all polyhydroxy acids and their lactones which include gluconic acid, gluconolactone, ribonolactone, galactonolactone, glucoheptonolactone, glucuronolactone and glucarolactone.

In the preparation of a urea composition comprising a stabilized molecular complex, urea preferably first is dissolved in water, and then a functional substance is slowly added to form a molecular complex. The formation of a molecular complex between the urea molecule and the functional substance is believed to be based on two intermolecular attractive forces, dipolar/dipolar and dipolar/ionic which are created between amino and/or carbonyl group of urea and hydroxyl and/or carboxyl group of the functional substance. More specifically, the dipolar/dipolar attractive force including hydrogen bonds preferably is created instantly between two amino and/or one carbonyl group of urea molecule and the hydroxyl group of the functional substance. The stronger dipolar/ionic attractive force is created between two amino and/or one carbonyl group of urea molecule and the carboxyl group of the functional substance.

When the functional substance is a polyhydroxy lactone such as gluconolactone or galactonolactone, some molecules of the lactone may react with water molecules to form a free acid, and the aqueous solution contains a mixture of lactone and free acid in equilibrium. In this case, the free acid is involved with dipolar/ionic and the lactone is involved with dipolar/dipolar attractive force with two amino and/or one carbonyl group of urea molecule. The formation of molecular complex is indicated by the change of pH, and the completion of the formation is shown by no more shifting in the pH of the solution. The molecular complex thus formed is quite stable for extended time of shelf life, and the stability is further enhanced by antioxidant property of polyhydroxy acids, lactones and certain hydroxyacids. After the molecular complex is formed, the solution may be adjusted to a desired pH up to 7.5 with an alkali. Once the molecular complex has been formed, urea composition can be formulated as solution, gel, cream, ointment or other cosmetically or dermatologically acceptable form. Since urea molecule in the molecular complex is controlled by two attractive forces, the release of urea molecule into the skin is under control-release mechanism for optimal therapeutic effects.

We have now discovered that urea compositions comprising a molecular complex are therapeutically effective for general care of skin, hair and nail; nasal, oral and vaginal mucosa; including treatment, healing and prevention of cosmetic conditions and dermatological indications as well as cosmetic and clinical signs of changes associated with intrinsic or extrinsic aging; the damages caused by extrinsic factors such as sunlight, air pollution, wind, cold, dampness, heat, chemicals, smoke, cigarette smoking, radiations including electromagnetic radiations and ionizing radiations. The urea compositions are also useful for reducing and soothing mucosa and skin erythema, inflammation or reaction caused by internal or external factors.

Urea compositions containing a molecular complex with a hydroxyacid or polyhydroxy acid have been found to be therapeutically effective for topical treatment of various cosmetic indications and dermatological disorders. Some examples are shown in Table 3.

TABLE 3
Therapeutic Effects of Urea Complex

| Subject | Urea Composition^{a} | Degree of Improvement |
|---|---|---|
| Male, age 12^{b} | Urea 15% | Minimal |
| | Urea Complex | 75% |
| Male, age 14^{b} | Urea 15% | 25% |
| | Urea Complex | 75% |
| | Urea Complex | 90% |
| Male, age 31^{b} | Urea 15% | minimal |
| | Urea Complex | 75% |
| | Urea Complex | 95-100% |
| Male, age 70^{c} | Urea 20% | minimal |
| | Urea Complex | 75% |
| Male, age 71^{d} | Urea Complex | 100% (itch) |
| | | 50% (eczema) |

^{a}Details in Examples
^{b}Severe dry skin conditions of ichthyosis
^{c}Hyperkeratotic calluses
^{d}Itchy nummular eczema

General cosmetic conditions and dermatological indications may be characterized as disturbed keratinization, inflammation, defective syntheses of dermal components, and changes associated with intrinsic and extrinsic aging of skin, nail and hair. Those conditions and indications include dryness or looseness of skin, nail and hair; xerosis; ichthyosis; palmar and plantar hyperkeratoses; uneven and rough surface of skin, nail and hair; dandruff; Darier's disease; lichen simplex chronicus; keratoses; acne; pseudofolliculitis barbae; dermatoses; eczema; psoriasis; pruritus; warts; herpes; age spots; lentigines; melasmas; blemished skin; hyperkeratoses; hyperpigmented skin; abnormal or diminished syntheses of collagen, glycosaminoglycans, proteoglycans and elastin as well as diminished levels of such components in the dermis; stretch marks; skin lines; fine lines; wrinkles; thinning of skin, nail plate and hair; skin thickening due to elastosis of photoaging, loss or reduction of skin, nail and hair resiliency, elasticity and recoilability; lack of skin, nail and hair lubricants and luster; dull and older-looking skin, nail and hair; fragility and splitting of nail and hair, or used as skin lightening.

Specific skin changes associated with aging include progressive thinning of skin, fragile skin, deepening of skin lines and fine lines, wrinkles including fine and coarse wrinkles, lusterless skin surface, coarse and uneven skin, loss of skin elasticity and recoilability, blemished and leathery skin, loss of skin lubricating substances, increased numbers of blotches and mottles, nodules, pre-cancerous lesions, pigmented spots and mottled skin, changes in qualities and quantities of collagen and elastic fibers, solar elastosis, decrease in collagen fibers, diminution in the number and diameter of elastic fibers in the papillary dermis, atrophy of the dermis, stretch marks, reduction in subcutaneous adipose tissue and deposition of abnormal elastic materials in the upper dermis, yellowing skin, telangiectatic skin and older-looking skin.

The functional substances of the present invention that can form a molecular complex with a urea molecule may be divided into two basic groups: (i) hydroxyacids; and (ii) polyhydroxy acids. The invention is not limited to these two basic groups, however, and any functional substance capable of forming a stable complex with urea to enable control release of the urea into integumental tissues can be used in the invention. Using the guidelines provided herein, those skilled in the art will be capable of designing a suitable and stable urea complex with any functional substance now known or later discovered.

Preferred hydroxyacids useful in the present invention include, but are not limited to, glycolic acid, 2-hydroxyisobutanoic acid, 2-hydroxybutanoic acid, 2-hydroxyoctanoic acid, 2-hydroxyeicosanoic acid, 2-hydroxytetraeicosanoic acid, mandelic acid, benzilic acid, 2-phenyl-2-hydroxypropanoic acid, 3-phenyl-2-hydroxypropanoic acid, tartronic acid, malic acid, tartaric acid, piscidic acid, citric acid, isocitric acid, homocitric acid, homoisocitric acid, agaricic acid, citramalic acid, 3-hydroxypropanoic acid, 3-hydroxybutanoic acid, 3-hydroxypentanoic acid, tropic acid, trethocanic acid, and mixtures and combinations thereof.

Preferred polyhydroxyacids useful in the present invention may be used as a free acid, an amide or as a lactone, and include, but are not limited to glyceric acid, pantoic acid, pantolactone, erythronic acid, mevalonic acid, mevalonolactone, isoascorbic acid, quinic acid, erythronolactone, threonic acid, threonolactone, ribonic acid, ribonolactone, arabinoic acid, arabinolactone, xylonic acid, xylonolactone, lyxonic acid, lyxonolactone, allonic acid, allonolactone, altronic acid, altronolactone, gluconic acid, gluconamide, gluconolactone, mannoic acid, mannolactone, gulonic acid, gulonolactone, idonic acid, idonolactone, galactonic acid, galactonolactone, talonic acid, talonolactone, glucoheptonic acid, glucoheptonolactone, glucaric acid, glucarolactone, galactaric acid, galactarolactone, glucuronic acid, glucuroamide, glucuronolactone, mannuronic acid, mannuronolactone, guluronic acid, guluronolactone, iduronic acid, iduronolactone, galacturonic acid, galacturonolactone, taluronic acid, taluronolactone, and mixtures and combinations thereof.

More generally, any hydroxyacid or polyhydroxyacid can be used in the invention. For example, the hydroxyacid may include alpha hydroxyacids and beta hydroxyacids, as well as ketoacids where the hydroxyl group is replaced by a keto group. The other useful acids are described in more detail below.

The alpha hydroxyacid is an organic carboxylic acid in which one hydroxyl group is attached to the alpha carbon of the acids. The generic structure of such alpha hydroxyacids may be represented as follows:

(Ra)(Rb)C(OH)COOH

where Ra and Rb are H, F, Cl, Br, I, alkyl, aralkyl or aryl group of saturated or unsaturated, isomeric or non-isomeric, straight or branched chain or cyclic form, having 1 to 25 carbon atoms, and in addition Ra and Rb may carry OH, CHO, COOH and alkoxyl group having 1 to 9 carbon atoms. The hydrogen atom attached to the carbon atom may be substituted by F, Cl, Br, I, or lower alkyl, aralkyl, aryl or alkoxyl group having 1 to 9 carbon atoms. The alpha hydroxyacids may be present as a free acid or lactone form, or in a partial salt form with an organic base or an inorganic alkali. The alpha hydroxyacids may exist as stereoisomers such as D, L, DL and meso forms.

When Ra and Rb are alkyl, they independently can be within any of the groups of C1-C5, C6-C10, C11-C 15, C16-C20, C21-C25 and C26-C29. Compounds within the invention thus include all of the possible combinations of Ra and Rb. Included within the foregoing is a subgenus of compounds having Ra and Rb independently selected from C1-C12.

Typical alkyl, aralkyl, aryl and alkoxyl groups for Ra and Rb include methyl, ethyl, propyl, propyl, isopropyl, butyl, pentyl, octyl, lauryl, stearyl, benzyl, phenyl, methoxyl, and ethoxyl. The alpha hydroxyacids of the first group may be subdivided into (1) alkyl alpha hydroxyacids, (2) aralkyl and aryl alpha hydroxyacids, (3) polyhydroxy alpha hydroxyacids, (4) polycarboxylic alpha hydroxyacids and (5) miscellaneous alpha hydroxyacids. The following are representative alpha hydroxyacids in each subgroup.

(1) Alkyl Alpha Hydroxyacids: 2-hydroxyethanoic acid (glycolic acid), 2-methyl 2-hydroxypropanoic acid (methyllactic acid), 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxyeicosanoic acid (alpha hydroxyarachidonic acid), 2-hydroxytetraeicosanoic acid (cerebronic acid), 2-hydroxytetraeicosenoic acid (alpha hydroxynervonic acid) and 2,4-dihydroxy-3,3-dimethylbutanoic acid (pantoic acid)

(2) Aralkyl And Aryl Alpha Hydroxyacids: 2-phenyl 2-hydroxyethanoic acid (mandelic acid); 2,2-diphenyl 2-hydroxyethanoic acid (benzilic acid), 3-phenyl 2-hydroxypropanoic acid (phenyllactic acid), 2-phenyl 2-methyl 2-hydroxyethanoic acid (atrolactic acid) and 4-hydroxymandelic acid.

(3) Polyhydroxy Alpha Hydroxyacids: 2,3-dihydroxypropanoic acid (glyceric acid); 2,3,4-trihydroxybutanoic acid (isomers; erythronic acid, threonic acid); 2,3,4,5-tetrahydroxypentanoic acid (isomers; ribonic acid, arabinoic acid, xylonic acid, lyxonic acid); 2,3,4,5,6-pentahydroxyhexanoic acid (isomers; allonic acid, altronic acid, gluconic acid, mannoic acid, gulonic acid, idonic acid, galactonic acid, talonic acid); 2,3,4,5,6,7-hexahydroxyheptanoic acid (isomers; glucoheptonic acid, galactoheptonic acid, mannoheptonic acid, etc.)

(4) Polycarboxylic Alpha Hydroxyacids: 2-hydroxypropane-1,3-dioic acid (tartronic acid); 2-hydroxybutane-1,4-dioic acid (malic acid); 2-hydroxy-2-methylbutane-1,4-dioic acid (citramalic acid); 2,3-dihydroxybutane-1,4-dioic acid (tartaric acid); 2,3,4-trihydroxypentane-1,5-dioic acid (isomers; ribaric acid, arabaric acid, xylaric acid, lyxaric acid); 2,3,4,5-tetrahydroxyhexane-1,6-dioic acid (isomers; glucaric acid, galactaric acid, mannaric acid, allaric acid, altraric acid, gularic acid, idaric acid, talaric acid); 2-hydroxy-1,2,3-propanetricarboxylic acid (citric acid); 1-hydroxy-1,2,3-propanetricarboxylic acid (isocitric acid); 1-hydroxy-1,2,4-butanetricarboxylic acid (homoisocitric acid); 2-hydroxy-3-hexadecyl-1,2,3-propanetricarboxylic acid (n-hexadecyl citric acid; agaricic acid).

(5) Miscellaneous Alpha Hydroxyacids: glyceruronic acid, erythruronic acid, threuronic acid; 2,3,4-trihydroxypentanuronic acids (isomers; riburonic acid, arabinuronic acid, xyluronic acid, lyxuronic acid); 2,3,4,5-tetrahydroxyhexanuronic acid (isomers; alluronic acid, altruronic acid, glucuronic acid, mannuronic acid, guluronic acid, iduronic acid, galacturonic acid, taluronic acid); 2,3,4,5,6-pentahydroxyheptanuronic acid (isomers; alloheptanuronic acid, altroheptanuronic acid, glucoheptanuronic acid, mannoheptanuronic acid, guloheptanuronic acid, idoheptanuronic acid, galactoheptanuronic acid, taloheptanuronic acid).

Other acids include related acids that are hydroxyacids in which the hydroxyl group is at any carbon position other than the alpha position, or the hydroxyl group is replaced by a keto group, or other miscellaneous organic hydroxycarboxylic acids which are not readily represented by a generic structure. For convenience, this group of compounds preferably is subdivided into (1) beta and other hydroxyacids, (2) alpha ketoacids, (3) miscellaneous compounds, and (4) oligomers and polymers of hydroxyacids.

(1) Beta and other hydroxyacids: These hydroxyacids have a hydroxyl group at any carbon position other than the alpha carbon positions. Most common one is the beta hydroxyacid. Representative hydroxyacids are as follows: 3-hydroxypropanoic acid (beta-hydroxypropanoic acid), 3-hydroxybutanoic acid (beta-hydroxybutyric acid), 2-phenyl-3-hydroxypropanoic acid (tropic acid); 3-hydroxy-3,7,11-trimethyldodecanoic acid (trethocanic acid) and 9,10,16-trihydroxyhexadecanoic acid (aleuritic acid).

(2) Alpha Ketoacids: Ketoacids are related to hydroxyacids in that the hydroxyl group is replaced by the keto group. Although the keto group can be at any position other than the terminal ends, the preferred one is an alpha ketoacid. For example, pyruvic acid, an alpha ketoacid is related to lactic acid in that the hydroxyl group of lactic acid is substituted by a keto group. In the skin, lactate dehydrogenase enzyme converts pyruvate to lactate and vice visa. The ketoacids have been found to have similar therapeutic effects as that of alpha hydroxyacids. The generic structure of alpha ketoacids may be represented as follows:

(Ra)COCOOH

wherein Ra is H, alkyl, aralkyl or aryl group of saturated or unsaturated, isomeric or non-isomeric, straight or branched chain or cyclic form, having 1 to 25 carbon atoms, and in addition Ra may carry F, Cl, Br, I, OH, CHO, COOH and alkoxyl group having 1 to 9 carbon atoms. The alpha ketoacids may be present as a free acid or in a salt form with an organic base or an inorganic alkali. The typical alkyl, aralkyl, aryl and alkoxyl groups for Ra include methyl, ethyl, propyl, isopropyl, butyl, pentyl, octyl, lauryl, stearyl, benzyl, phenyl, methoxyl and ethoxyl.

Representative alpha ketoacids which may be useful for cosmetic conditions and dermatologic indications are listed below: 2-ketoethanoic acid (glyoxylic acid), 2-ketopropanoic acid (pyruvic acid), 2-phenyl-2-ketoethanoic acid (benzoylformic acid), 3-phenyl-2-ketopropanoic acid (phenylpyruvic acid), 2-ketobutanoic acid, 2-ketopentanoic acid, 2-ketohexanoic acid, 2-ketoheptanoic acid, 2-ketooctanoic acid and 2-ketododecanoic acid.

(3) Miscellaneous Hydroxyacids: These hydroxyacids have similar therapeutic effects as that of alpha hydroxyacids but their chemical structures are not readily represented by the foregoing generic structures. These compounds are listed as follows: quinic acid (1,3,4,5-tetrahydroxycyclohexanecarboxylic acid), piscidic acid (4-hydroxybenzyltartaric acid), isoascorbic acid (D-erythro-hex-2-enonic acid-lactone), 2-hexulosonic acids (isomers; arabino-2-hexulosonicacid, xylo-2-hexulosonic acid, ribo-2-hexulosonic acid, lyxo-2-hexulosonic acid), 5-hexulosonic acids (isomers; arabino-5-hexulosonic acid, xylo-5-hexulosonic acid, ribo-5-hexulosonic acid, lyxo-5-hexulosonic acid).

(4) Oligomers of Hydroxyacids: When two or more molecules of hydroxyacids either identical or non-identical are reacted chemically to each other, oligomers are formed. The chemical bond is usually an ester bond formed from the carboxyl group of one monomer and the hydroxyl group of a second monomer by eliminating a water molecule. In general, oligomers consist of 2 to 10 monomers of hydroxyacids. The oligomers may be cyclic or non-cyclic form or a mixture of the two. The generic structure of oligomers of hydroxyacids may be described as follows.

(AHA)m-n(H₂O)

wherein, AHA is a hydroxyacid described above, m=2-10, with a preferred number of 2-4, and n=m-1. AHA in each monomer may be identical or not identical. For example, glycolyl glycolate, glycolyl lactate, lactyl lactate and lactyl glycolate. Representative oligomers of AHA are listed below: glycolyl glycolate, lactyl lactate, citryl citrate, glycolyl citrate, citryl glycolate, lactyl citrate, citryl lactate, malyl malate, malyl glycolate, tartaryl tartrate, tartaryl glycolate, glycolyl tartrate, glycolyl glycolyl glycolate, lactyl lactyl lactate, and other AHA oligomers.

The alpha hydroxyacids and related acids may exist as free acid, partial salt and lactone forms. A partial salt is formed when an alpha hydroxyacid or related acid is partially neutralized with an organic or inorganic alkali. For example, glycolic acid 1 mole is reacted with ammonium hydroxide 0.5 mole. The reaction mixture thus formed consists of glycolic free acid 0.5 mole and ammonium glycolate 0.5 mole. When citric acid 1 mole is reacted with sodium hydroxide 1 mole the reaction mixture thus formed consists of citric acid monosodium salt 1 mole. Since citric acid has three carboxylic acid groups per molecule citric acid monosodium salt is a partial salt containing two free carboxylic acid groups and is still very acidic in nature.

Many alpha hydroxyacids and related acids may form intramolecular lactones. Some examples include gluconolactone, galactonolactone, glucuronolactone, galacturonolactone, gulonolactone, ribonolactone, saccharic acid lactone, pantoyllactone, glucoheptonolactone, mannonolactone, and galactoheptonolactone. All of the above described hydroxyacids, polyhydroxyacids, keto acids, and related acids may be used in the present invention. These functional substances may be used alone, or in various combinations. For example, two or more hydroxyacids may be used to form a complex with urea.

The urea composition of the invention that includes the molecular complex may also incorporate other cosmetic, pharmaceutical or topical agents to further expand the utilities for maximal therapeutic efficacies. The cosmetic, pharmaceutical and other topical agents that may be incorporated into the urea compositions include those that improve or eradicate age spots, keratoses and wrinkles; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotionsickness agents; antiinflammatory agents; antihyperkeratolytic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; vitamins; corticosteroids; tanning agents; humectants; hormones; retinoids; gum disease or oral care agents; topical cardiovascular agents; corn, callus and wart removing agents; dipilating agents; and other dermatologicals.

Some examples of cosmetic, pharmaceutical and other topical agents are aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascoryl palimate, atropine, azelaic acid, bacitracin, bemegride, beclomethasone dipropionate, benzophenone, benzoyl peroxide, betamethasone dipropionate, betamethasone valerate, brompheniramine, bupivacaine, butoconazole, calcipotriene, camphor, capsaicin, carbamide peroxide, chitosan, chlorhexidine, chloroxylenol, chlorpheniramine, ciclopirox, clemastine, clindamycin, clioquinol, clobetasol propionate, clotrimazole, coal tar, cromolyn, crotamiton, cycloserine, dehydroepiandrosterone, desoximetasone, dexamethasone, diphenhydramine, doxypin, doxylamine, dyclonine, econazole, erythromycin, estradiol, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, ichthammol, imiquimod, indomethacin, ketoconazole, ketoprofen, kojic acid, lidocaine, meclizine, meclocycline, menthol, mepivacaine, methyl nicotinate, methyl salicylate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocaine, procaine, promethazine propionate, propranolol, pseudoephedrine, pyrethrin, pyrilamine, resorcinol, retinal, 13-cis retinoic acid, retinoic acid, retinol, retinyl acetate, retinyl palmitate, salicylamide, salicylic acid, selenium sulfide, shale tar, sulconazole, sulfur, sulfadiazine, tazarotene, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, vitamin E acetate, wood tar, and zinc pyrithione.

Other examples of cosmetic or other agents that may be combined with urea compositions include other organic hydroxycarboxylic acids, ketoacids and related compounds. Examples of such acids are described in U.S. Pat. Nos. 5,422,370, 5,547,988, 5,470,880, 5,385,938, and 5,877,212, each of which is incorporated by reference herein in its entirety.

Yet another example of cosmetic or other agents that may be combined with the urea compositions include N-acetyl aldosamines, N-acetylamino acids, and related N-acetyl compounds as described in U.S. Pat. No. 6,159,485, the disclosure of which is incorporated by reference herein in its entirety. Representative examples of such compounds include N-acetyl-L-proline, N-acetyl-L-glutamine, N-acetyl-L-lysine, N-acetyl-L-cysteine and N-acetyl-glycine.

An additional example of cosmetic or other agents that may be incorporated with the inventive urea compositions include oligosaccharide aldonic acids as described in U.S. Pat. No. 6,335,023, the disclosure of which is incorporated by reference herein in its entirety. Representative examples of such compounds include lactobionic acid, maltobionic acid and cellobionic acid.

Urea compositions comprising a molecular complex with a functional substance can be formulated as solution, gel, lotion, cream, ointment, shampoo, spray, stick, powder, masque, mouth rinse or wash, vaginal gel or other form acceptable for use on skin, nail, hair, oral mucosa, vaginal mucosa, mouth or gums.

To prepare a solution composition, urea preferably is first dissolved in water, and then a functional substance such as hydroxyacid or polyhydroxy acid is slowly added to form a molecular complex. The formation of the molecular complex is complete as shown by no more change in pH of the solution. Other solvents such as ethanol, propylene glycol, butylene glycol, and other topically acceptable vehicle may be added. The concentration of urea ranges from about 0.1 to about 80% and that of functional substance ranges from about 0.1 to about 70% by weight of the total concentration. The preferred concentration of urea is from about 2 to about 50% and that of functional substance is from about 1 to 30% by weight of the total composition. It is even more preferred that the concentration of urea range from about 15% to about 40%, and most preferably from about 18% to about 25%, by weight based on the total weight of the composition. It is even more preferred that the concentration of functional substance range from about 2% to about 15%, and most preferably from about 2% to about 6%, by weight based on the total weight of the composition.

To prepare a urea complex-containing composition of the invention, whereby the molecular complex is in lotion, cream or ointment form, a functional substance preferably is first added to a urea solution to form a molecular complex as described above. The complex solution thus formed then preferably is mixed with a desired base or pharmaceutically acceptable vehicle to make lotion, cream or ointment. The respective concentrations of urea and the functional substance are the same as described above.

A topical composition of the instant invention also may be formulated in a gel or shampoo form. A typical gel composition can be formulated by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate to a molecular complex comprising urea and a functional substance. The preferred concentration of the gelling agent may range from 0.1 to 4 percent by weight of the total composition. In the preparation of a shampoo, a molecular complex comprising urea and a functional substance preferably is mixed with a shampoo base. Concentrations of urea and the functional substance used in gel or shampoo form are the same as described above.

To prepare a combination composition for enhanced effects or expanded utilities, a cosmetic, pharmaceutical or other topical agent can be incorporated into any one of the above formulations. The composition may contain other additives and excipients, as will be appreciated to those skilled in the art. Other forms of compositions for delivery of a molecular complex comprising urea and a functional substance of the instant invention are readily blended, prepared or formulated by those skilled in the art.

The following are illustrative examples of formulations and testings according to this invention. Although the examples utilize only selected compounds and formulations, it should be understood that the following examples are illustrative and not limited. Therefore, any of the aforementioned functional substances may be substituted according to the teachings of this invention in the following examples.

### EXAMPLE 1

Study on shelf life stability of 20% urea was carried out as follows. Urea 20% solution was prepared by dissolving urea 20 g in water 80 ml. The solution had a pH of 7.4. After 11 months at room temperature, urea 20% solution had a pH change of from 7.4 to 8.8. The result shows that urea 20% solution is not chemically stable for storage or shelf life.

### EXAMPLE 2

Study on shelf life stability of 40% urea was carried out as follows. Urea 40% solution was prepared by dissolving urea 40 g in water 60 ml. The solution had a pH of 7.4. After 11 months at room temperature, urea 40% solution had a pH change of from 7.4 to 9.0. The result shows that urea 40% solution is not chemically stable for storage or shelf life.

### EXAMPLE 3

Study on shelf life stability of an inventive composition containing urea 20% solution was carried out as follows. Urea 20 g was dissolved in water 65 ml and glycolic acid 5 g was slowly added to form a molecular complex until the solution changed pH from 7.4 to a pH of 2.4. L-Arginine 10 g then was added to raise the pH to 8.2. After 11 months at room temperature, the inventive urea 20% solution had a pH 8.0. The result shows that the inventive urea 20% solution is chemically stable for storage or shelf life.

### EXAMPLE 4

A typical urea composition comprising a molecular complex with a functional substance was prepared as follows. Urea 40 g was dissolved in water 55 ml and gluconolactone 5 g was slowly added to form a molecular complex. The formation of the molecular complex was completed when the solution changed pH from 7.4 to 2.9. A clear solution comprising the molecular complex had a pH 2.9, and contained 40% urea and 5% gluconolactone. The pH of the solution could be optionally raised to pH 7.0 by the addition of 2 g L-arginine into the solution.

### EXAMPLE 5

Urea 40 g was dissolved in water 56 ml and glucoheptonolactone 4 g was slowly added to form a molecular complex until the pH changed from 7.4 to 4.4. A clear solution comprising the molecular complex had pH 4.4, and contained 40% urea and 4% glucoheptonolactone.

### EXAMPLE 6

Urea 20 g was dissolved in water 78 ml and ribonolactone 2 g was slowly added to form a molecular complex until pH changed from 7.4 to 4.2. A clear solution containing the molecular complex had pH 4.2, and contained 20% urea and 2% ribonolactone.

### EXAMPLE 7

Urea 20 g was dissolved in water 78 ml and mandelic acid 2 g was slowly added to form a molecular complex until pH changed from 7.4 to 2.4. A clear solution containing the molecular complex had pH 2.4, and contained 20% urea and 2% mandelic acid.

### EXAMPLE 8

Urea 20 g was first dissolved in water 27.5 ml and gluconolactone 2.5 g was slowly added to form a molecular complex until pH changed from 7.4 to 3.3. A clear solution containing the molecular complex was mixed with hydrophilic ointment or oil-in-water cream base 50 g. The white cream thus formulated had pH 2.9, and contained 20% urea and 2.5% gluconolactone.

### EXAMPLE 9

Urea 20 g was first dissolved in water 25 ml and galactonolactone 5 g was slowly added to form a molecular complex until the solution changed pH from 7.4 to 4.7. A clear solution containing the molecular complex was mixed with hydrophilic ointment or oil-in-water cream base 50 g. The white cream thus obtained had pH 4.7, and contained 20% urea and 5% galactonolactone.

### EXAMPLE 10

Urea 25 g was dissolved in water 25 ml and glucuronolactone 6 g was slowly added to form a molecular complex until the solution changed pH from 7.4 to 3.8. A clear solution containing the molecular complex was mixed with hydrophilic ointment or oil-in-water cream base 44 g. The white cream thus obtained had pH 3.8, and contained 25% urea and 6% glucuronolactone.

### EXAMPLE 11

Urea 30 g was dissolved in water 25 ml and erythronolactone 7 g was slowly added to form a molecular complex until the solution changed pH from 7.4 to 3.5. A clear solution containing the molecular complex was mixed with hydrophilic ointment or oil-in-water cream base 38 g. The white cream thus obtained had pH 3.5, and contained 30% urea and 7% erythronolactone.

### EXAMPLE 12

Urea 15 g was dissolved in water 25 ml and gulonolactone 5 g was slowly added to form a molecular complex until the solution changed pH from 7.4 to 3.0. A clear solution containing the molecular complex was mixed with hydrophilic ointment or oil-in-water cream base 55 g. The white cream thus obtained had pH 3.0, and contained 15% urea and 5% gulonolactone.

### EXAMPLE 13

Urea 10 g was dissolved in water 25 ml and glyceric acid 40% in water solution 12.5 g was slowly added to form a molecular complex until the solution changed pH from 7.4 to 2.3. A clear solution containing the molecular complex was mixed with hydrophilic ointment or oil-in-water cream base 52.5 g. The white cream thus obtained had pH 2.3, and contained 10% urea and 5% glyceric acid.

### EXAMPLE 14

Urea 20 g was dissolved in water 25 ml and isocitric acid lactone 5 g was slowly added to form a molecular complex until the solution changed pH from 7.4 to 1.9. A clear solution containing the molecular complex was mixed with hydrophilic ointment or oil-in-water cream base 50 g. The white cream thus obtained had pH 1.9, and contained 20% urea and 5% isocitric acid lactone.

### EXAMPLE 15

Urea 50 g was dissolved in water 48 ml and isoascorbic acid 2 g was slowly added to form a molecular complex until the solution changed pH from 7.4 to 3.0. The clear solution containing the molecular complex had pH 3.0, and contained 50% urea and 2% isoascorbic acid.

### EXAMPLE 16

Urea 20 g was dissolved in water 53 ml and glucuronamide 5 g was slowly added to form a molecular complex until the solution changed pH from 7.4 to 3.3. Propylene glycol 22 ml was added to the solution containing the molecular complex. A clear solution thus obtained had pH 3.3, and contained 20% urea and 5% glucuronamide in molecular complex created by dipolar/dipolar attractive force.

### EXAMPLE 17

Urea 25 g was dissolved in water 50 ml and tartronic acid 4 g was slowly added to form a molecular complex until the solution changed pH from 7.4 to 1.8. Propylene glycol 21 ml was added to the solution. A clear solution thus obtained had pH 1.8, and contained 25% urea and 4% tartronic acid.

### EXAMPLE 18

Urea 30 g was dissolved in warm water 30 ml and pantolactone 7 g was slowly added to form a molecular complex until the solution changed from pH 7.4 to 4.1. A clear solution containing the molecular complex was mixed with hydrophilic ointment or oil-in-water cream base 33 g. The white cream thus obtained had pH 4.1, and contained 30% urea and 7% pantolactone.

### EXAMPLE 19

Urea 20 g was dissolved in water 25 ml and glucarolactone (saccharic acid lactone) 5 g was slowly added to form a molecular complex until the solution changed pH from 7.4 to 2.2. A clear solution containing the molecular complex was mixed with hydrophilic ointment or oil-in-water cream base 50 g. The white cream thus obtained had pH 2.2, and contained 20% urea and 5% glucarolactone.

### EXAMPLE 20

Urea 10 g was dissolved in water 34 ml and quinic acid 6 g was slowly added to form a molecular complex until the solution changed pH from 7.4 to 1.9. A clear solution containing the molecular complex was mixed with hydrophilic ointment or oil-in-water cream base 50 g. The white cream thus obtained had pH 1.9, and contained 10% urea and 6% quinic acid.

### EXAMPLE 21

Urea 15 g was dissolved in water 27 ml and galacturonic acid 8 g was slowly added to form a molecular complex until the solution changed pH from 7.4 to 1.9. A clear solution containing the molecular complex was mixed with hydrophilic ointment or oil-in-water cream base 50 g. The white cream thus obtained had pH 1.9, and contained 15% urea and 8% galacturonic acid.

### EXAMPLE 22

A comparative study on severe dry skin condition was carried out as follows. Urea 15 g was dissolved in water 15 ml and the solution thus obtained was mixed with a cream base 70 g. This cream containing 15% urea was used as one of the two control vehicles. Another control vehicle containing 5% glycolic acid was prepared from glycolic acid 5 g, water 5 ml and a cream base 90 g. In addition, an inventive urea 15% composition was prepared from urea 15 g as a molecular complex with glycolic acid 5 g.

A male subject, age 31, having severe dry skin condition of lamellar ichthyosis topically applied twice daily the above three creams on three test sites of his left upper arm. After 3 weeks, the urea alone cream gave minimal improvement, glycolic acid alone cream gave 25% improvement and the inventive urea cream containing the molecular complex with glycolic acid provided 75% improvement of the severe dry skin condition. This result shows that the inventive urea composition containing a molecular complex is therapeutically more effective than urea or hydroxyacid alone formulation.

### EXAMPLE 23

A comparative study on hyperkeratotic calluses was carried out as follows. Urea 20% alone cream was prepared from urea 20 g, water 20 ml and a cream base 60 g. Mandelic acid 5% alone cream was prepared from mandelic acid 5 g, water 10 ml and a cream base 85 g. An inventive urea 20% composition containing a molecular complex with 5% mandelic acid was prepared from urea 20 g, water 25 ml and mandelic acid 5 g in a cream base 50 g. A male subject, age 70, having hyperkeratotic calluses on his both feet topically applied once daily urea 20% cream on his left foot and 5% mandelic acid cream on his right foot. After one week, the left foot had minimal improvement on the calluses, and the right foot had 25% improvement on hyperkeratotic calluses. The inventive 20% urea cream containing a molecular complex with 5% mandelic acid was then topically applied once daily to his left foot. After one week, his left foot had 75% improvement on the hyperkeratotic calluses. The treated skin appeared smooth without fissures.

### EXAMPLE 24

Another comparative study on severe dry skin condition was carried out as follows. Urea 15% alone cream was prepared from urea 15 g, water 15 ml and a cream base 70 g. An inventive urea 15% cream containing a molecular complex with 5% mandelic acid was prepared from urea 15 g, water 20 ml and mandelic acid 5 g in a cream base 60 g.

A male subject, age 14, having severe dry skin condition of lamellar ichthyosis topically applied twice daily the urea alone 15% cream on his right elbow and the inventive urea 15% cream containing the molecular complex with mandelic acid 5% on his left elbow. After 5 days, the right elbow gave 25% improvement but the left elbow had 75% improvement. This result shows that the inventive urea composition containing the molecular complex is therapeutically more effective than urea alone for topical treatment of severe dry skin conditions.

### EXAMPLE 25

Another comparative study on severe dry skin condition was carried out as follows. Urea alone 15% cream was prepared from urea 15 g, water 15 ml and a cream base 70 g. An inventive urea 15% cream containing a molecular complex was prepared from urea 15 g, glycolic acid 5 g, mandelic acid 5 g, water 25 ml and a cream base 50 g.

A male subject, age 12, having severe dry skin condition of lamellar ichthyosis topically applied twice daily urea alone 15% cream on his right elbow and the inventive urea 15% cream on his left elbow. After one week, while his right elbow gave minimal improvement, his left elbow gave 75% improvement. This result shows that the inventive urea composition containing a molecular complex is therapeutically more effective than urea alone formulation for topical treatment of severe dry skin condition.

### EXAMPLE 26

Another comparative study on severe dry skin condition was carried out as follows. Mandelic acid alone 5% cream was prepared from mandelic acid 5 g, water 10 ml and a cream base 85 g. An inventive urea composition containing a molecular complex was prepared from urea 15 g, mandelic acid 5 g, glycolic acid 5 g, N-acetyl-L-cysteine 2 g, vitamin E acetate 1 g and retinyl acetate 0.5 g, water 25 ml and a cream base 46.5.

A male subject, age 14, having severe dry skin condition of lamellar ichthyosis topically applied twice daily mandelic acid alone 5% cream on his right knee and the inventive urea composition on his left knee. After 5 days, while his right knee showed 50% improvement, his left knee gave 95% improvement. This result reveals that the inventive urea composition containing a molecular complex is therapeutically effective for topical treatment of severe dry skin condition.

### EXAMPLE 27

An inventive urea composition containing a molecular complex was prepared from urea 15 g, mandelic acid 6 g, glycolic acid 3.5 g, N-acetyl-L-cysteine 1 g, vitamin E acetate 1 g and retinyl acetate 0.5 g, water 27 ml and a cream base 46.

A male subject, age 31, having severe dry skin condition of lamellar ichthyosis topically applied twice daily the above inventive urea cream to whole body except the back. After 16 days, the treated areas of skin became nearly normal and the improvement had been clinically judged to be 95 to 100%. This result reveals that the inventive urea composition containing a molecular complex is therapeutically effective for topical treatment of severe dry skin condition.

### EXAMPLE 28

Topical urea compositions containing a molecular complex and other topical agent(s) were also prepared as follows. In one preparation, urea 10 g was dissolved in water 25 ml and the solution had pH 7.1. Gluconolactone 10 g was added to form a molecular complex until the solution changed pH from 7.1 to 2.3. Propylene glycol 5 ml was added and L-Arginine 1 g was added to adjust the solution to pH 3.1. Diphenhydramine base 2 g and N-acetyl-L-proline 1.7 g in 10 ml aqueous solution were added and the solution was mixed with a cream base to make total weight of 100 g. Cream A thus formulated had pH 3.1 and contained 10% urea complex with 10% gluconolactone, 2% diphenhydramine and 1.7% N-acetyl-L-proline. Cream B was prepared from Cream A by the addition of 0.4% hydrocortisone-17-valerate.

A male subject, age 71, having itchy eczema lesions on both forearms topically applied twice daily Cream A on the left forearm and Cream B on the right forearm. After a few minutes, the itch on both forearms disappeared completely and stayed free of itch for the next 8 hours. After one week of topical application, eczema lesions on both forearms had more than 50% improvement. These results show that urea compositions containing a molecular complex and other topical agent(s) are therapeutically effective for topical treatment of inflammatory diseases.

Other embodiments, uses, and advantages of the invention will be apparent to those skilled in the art from consideration of the specification and practice of the invention disclosed herein. The specification should be considered exemplary only, and the scope of the invention is accordingly intended to be supplemented by the following claims. The scope of the invention covers any compositions comprising urea and a functional substance without stating that a molecular complex has been formed because the formation of such complex will occur based on scientific principles and observations.

What is claimed is:

1. A composition comprising a molecular complex formed between urea and a functional substance comprising at least one hydroxyl group and one carboxyl group either as a free acid, salt, an amide or a lactone.

2. The composition of claim 1, wherein the functional substance is a hydroxyacid or polyhydroxy acid.

3. The composition of claim 2, wherein the hydroxyacid is selected from the group consisting of glycolic acid, 2-hydroxyisobutanoic acid, 2-hydroxybutanoic acid, 2-hydroxyoctanoic acid, 2-hydroxyeicosanoic acid, 2-hydroxytetraeicosanoic acid, mandelic acid, benzilic acid, 2-phenyl-2-hydroxypropanoic acid, 3-phenyl-2-hydroxypropanoic acid, tartronic acid, malic acid, tartaric acid, piscidic acid, citric acid, isocitric acid, homocitric acid, homoisocitric acid, agaricic acid, citramalic acid, 3-hydroxypropanoic acid, 3-hydroxybutanoic acid, 3-hydroxypentanoic acid, tropic acid, trethocanic acid, and mixtures and combinations thereof.

4. The composition of claim 2, wherein the hydroxyacid is glycolic acid.

5. The composition of claim 2, wherein the polyhydroxyacid is selected from the group consisting of glyceric acid, pantoic acid, pantolactone, erythronic acid, mevalonic acid, mevalonolactone, isoascorbic acid, quinic acid, erythronolactone, threonic acid, threonolactone, ribonic acid, ribonolactone, arabinoic acid, arabinolactone, xylonic acid, xylonolactone, lyxonic acid, lyxonolactone, allonic acid, allonolactone, altronic acid, altronolactone, gluconic acid, gluconamide, gluconolactone, mannoic acid, mannolactone, gulonic acid, gulonolactone, idonic acid, idonolactone, galactonic acid, galactonolactone, talonic acid, talonolactone, glucoheptonic acid, glucoheptonolactone, glucaric acid, glucarolactone, galactaric acid, galactarolactone, glucuronic acid, glucuroamide, glucuronolactone, mannuronic acid, mannuronolactone, guluronic acid, guluronolactone, iduronic acid, iduronolactone, galacturonic acid, galacturonolactone, taluronic acid, taluronolactone, and mixtures and combinations thereof.

6. The composition of claim 2, wherein the polyhydroxy acid is gluconic acid or gluconolactone.

7. The composition of claim 1, wherein the concentration of urea is within the range of from about 0.1 to about 80% by weight, based on the total weight of the composition.

8. The composition of claim 7, wherein the concentration of urea is within the range of from about 2 to about 50% by weight, based on the total weight of the composition.

9. The composition of claim 7, wherein the concentration of urea is within the range of from about 15 to about 40% by weight, based on the total weight of the composition.

10. The composition of claim 7, wherein the concentration of urea is within the range of from about 18 to about 25% by weight, based on the total weight of the composition.

11. The composition of claim 1, wherein the concentration of the functional substance is within the range of from about 0.1 to about 70% by weight, based on the total weight of the composition.

12. The composition of claim 11, wherein the concentration of the functional substance is within the range of from about 1 to about 30% by weight, based on the total weight of the composition.

13. The composition of claim 11, wherein the concentration of the functional substance is within the range of from about 2 to about 15% by weight, based on the total weight of the composition.

14. The composition of claim 11, wherein the concentration of the functional substance is within the range of from about 2 to about 6% by weight, based on the total weight of the composition.

15. The composition of claim 1, further comprising at least one of a cosmetic, a pharmaceutical, or other topical agents.

16. The composition of claim 15, wherein the cosmetic, pharmaceutical and other topical agents comprise one or more ingredient selected from the group consisting of: compounds that improve or eradicate age spots, keratoses and wrinkles; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotionsickness agents; antiinflammatory agents; antihyperkeratolytic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; vitamins; corticosteroids; tanning agents; humectants; hormones; retinoids; gum disease or oral care agents; topical cardiovascular agents; corn, callus and wart removing agents; dipilating agents; and other dermatologicals.

17. The composition of claim 16, wherein the cosmetic, pharmaceutical and other topical agents comprise one or more ingredient selected from the group consisting of aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascoryl palimate, atropine, azelaic acid, bacitracin, bemegride, beclomethasone dipropionate, benzophenone, benzoyl peroxide, betamethasone dipropionate, betamethasone valerate, brompheniramine, bupivacaine, butoconazole, calcipotriene, camphor, capsaicin, carbamide peroxide, chitosan, chlorhexidine, chloroxylenol, chlorpheniramine, ciclopirox, clemastine, clindamycin, clioquinol, clobetasol propionate, clotrimazole, coal tar, cromolyn, crotamiton, cycloserine, dehydroepiandrosterone, desoximetasone, dexamethasone, diphenhydramine, doxypin, doxylamine, dyclonine, econazole, erythromycin, estradiol, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, ichthammol, imiquimod, indomethacin, ketoconazole, ketoprofen, kojic acid, lidocaine, meclizine, meclocycline, menthol, mepivacaine, methyl nicotinate, methyl salicylate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocaine, procaine, promethazine propionate, propranolol, pseudoephedrine, pyrethrin, pyrilamine, resorcinol, retinal, 13-cis retinoic acid, retinoic acid, retinol, retinyl acetate, retinyl palmitate, salicylamide, salicylic acid, selenium sulfide, shale tar, sulconazole, sulfur, sulfadiazine, tazarotene, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, vitamin E acetate, wood tar, zinc pyrithione, and mixtures thereof.

18. The composition as claimed in claim 1, wherein the composition is in the form of a solution, gel, lotion, cream, ointment, shampoo, spray, stick, powder, masque, mouth rinse or wash, vaginal gel or other form acceptable for use on skin, nail, hair, oral mucosa, vaginal mucosa, mouth or gums.

19. A method of making a composition comprising a complex of urea and a functional substance comprising at least one hydroxyl group and one carboxyl group either as a free acid, an amide or a lactone, the method comprising forming a complex between urea and the functional substance, and formulating the complex into a topically acceptable vehicle.

20. The method as claimed in claim 19, wherein the composition is in the form of a solution, gel, lotion, cream, ointment, shampoo, spray, stick, powder, masque, mouth rinse or wash, vaginal gel or other form acceptable for use on skin, nail, hair, oral mucosa, vaginal mucosa, mouth or gums.

21. The method as claimed in claim 20, wherein the composition is a solution.

22. The method as claimed in claim 21, wherein the solution is formed by dissolving urea in water, slowly adding to the solution the functional substance to form a molecular complex.

23. The method as claimed in claim 20, wherein the composition is selected from the group consisting of a lotion, cream, and ointment.

24. The method as claimed in claim 23, wherein the lotion, cream, or ointment is formed by adding the functional substance to a urea solution to form a molecular complex, and then mixing the complex solution with a topically or pharmaceutically acceptable vehicle.

25. The method as claimed in claim 20, wherein the composition is a gel.

26. The method as claimed in claim 25, wherein the gel is formed adding the functional substance to a urea solution to form a molecular complex, and then mixing the complex solution with a gelling agent.

27. The method as claimed in claim 26, wherein the gelling agent is selected from the group consisting of chitosan, methyl cellulose, ethyl cellulose, polyvinyl alcohol, polyquaterniums, hydroxyethylcellulose, hydroxypropylcellulose, hydroxypropylmethylcellulose, carbomer, ammoniated glycyrrhizinate, and mixtures thereof.

28. The method of claim 19, wherein the functional substance is a hydroxyacid or polyhydroxy acid.

29. The method of claim 28, wherein the hydroxyacid is selected from the group consisting of glycolic acid, 2-hydroxyisobutanoic acid, 2-hydroxybutanoic acid, 2-hydroxyoctanoic acid, 2-hydroxyeicosanoic acid, 2-hydroxytetraeicosanoic acid, mandelic acid, benzilic acid, 2-phenyl-2-hydroxypropanoic acid, 3-phenyl-2-hydroxypropanoic acid, tartronic acid, malic acid, tartaric acid, piscidic acid, citric acid, isocitric acid, homocitric acid, homoisocitric acid, agaricic acid, citramalic acid, 3-hydroxypropanoic acid, 3-hydroxybutanoic acid, 3-hydroxypentanoic acid, tropic acid, trethocanic acid, and mixtures and combinations thereof.

30. The method of claim 28, wherein the hydroxyacid is glycolic acid.

31. The method of claim 28, wherein the polyhydroxyacid is selected from the group consisting of glyceric acid, pantoic acid, pantolactone, erythronic acid, mevalonic acid, mevalonolactone, isoascorbic acid, quinic acid, erythronolactone, threonic acid, threonolactone, ribonic acid, ribonolactone, arabinoic acid, arabinolactone, xylonic acid, xylonolactone, lyxonic acid, lyxonolactone, allonic acid, allonolactone, altronic acid, altronolactone, gluconic acid, gluconamide, gluconolactone, mannoic acid, mannolactone, gulonic acid, gulonolactone, idonic acid, idonolactone, galactonic acid, galactonolactone, talonic acid, talonolactone, glucoheptonic acid, glucoheptonolactone, glucaric acid, glucarolactone, galactaric acid, galactarolactone, glucuronic acid, glucuroamide, glucuronolactone, mannuronic acid, mannuronolactone, guluronic acid, guluronolactone, iduronic acid, iduronolactone, galacturonic acid, galacturonolactone, taluronic acid, taluronolactone, and mixtures and combinations thereof.

32. The method of claim 28, wherein the polyhydroxy acid is gluconic acid or gluconolactone.

33. A method of treating a cosmetic or dermatological disorder comprising topically applying to an area of the skin containing the cosmetic or dermatological disorder, a therapeutically effective amount of a composition comprising a molecular complex formed between urea and a functional substance comprising at least one hydroxyl group and one carboxyl group either as a free acid, an amide or a lactone.

34. The method of claim 33, wherein the functional substance is a hydroxyacid or polyhydroxy acid.

35. The method of claim 34, wherein the hydroxyacid is selected from the group consisting of glycolic acid, 2-hydroxyisobutanoic acid, 2-hydroxybutanoic acid, 2-hydroxyoctanoic acid, 2-hydroxyeicosanoic acid, 2-hydroxytetraeicosanoic acid, mandelic acid, benzilic acid, 2-phenyl-2-hydroxypropanoic acid, 3-phenyl-2-hydroxypropanoic acid, tartronic acid, malic acid, tartaric acid, piscidic acid, citric acid, isocitric acid, homocitric acid, homoisocitric acid, agaricic acid, citramalic acid, 3-hydroxypropanoic acid, 3-hydroxybutanoic acid, 3-hydroxypentanoic acid, tropic acid, trethocanic acid, and mixtures and combinations thereof.

36. The method of claim 34, wherein the hydroxyacid is glycolic acid.

37. The method of claim 34, wherein the polyhydroxyacid is selected from the group consisting of glyceric acid, pantoic acid, pantolactone, erythronic acid, mevalonic acid, mevalonolactone, isoascorbic acid, quinic acid, erythronolactone, threonic acid, threonolactone, ribonic acid, ribonolactone, arabinoic acid, arabinolactone, xylonic acid, xylonolactone, lyxonic acid, lyxonolactone, allonic acid, allonolactone, altronic acid, altronolactone, gluconic acid, gluconamide, gluconolactone, mannoic acid, mannolactone, gulonic acid, gulonolactone, idonic acid, idonolactone, galactonic acid, galactonolactone, talonic acid, talonolactone, glucoheptonic acid, glucoheptonolactone, glucaric acid, glucarolactone, galactaric acid, galactarolactone, glucuronic acid, glucuroamide, glucuronolactone, mannuronic acid, mannuronolactone, guluronic acid, guluronolactone, iduronic acid, iduronolactone, galacturonic acid, galacturonolactone, taluronic acid, taluronolactone, and mixtures and combinations thereof.

38. The method of claim 34, wherein the polyhydroxy acid is gluconic acid or gluconolactone.

39. The method of claim 33, wherein the concentration of urea is within the range of from about 0.1 to about 80% by weight, based on the total weight of the composition.

40. The method of claim 39, wherein the concentration of urea is within the range of from about 2 to about 50% by weight, based on the total weight of the composition.

41. The method of claim 39, wherein the concentration of urea is within the range of from about 15 to about 40% by weight, based on the total weight of the composition.

42. The method of claim 39, wherein the concentration of urea is within the range of from about 18 to about 25% by weight, based on the total weight of the composition.

43. The method of claim 33, wherein the concentration of the functional substance is within the range of from about 0.1 to about 70% by weight, based on the total weight of the composition.

44. The method of claim 43, wherein the concentration of the functional substance is within the range of from about 1 to about 30% by weight, based on the total weight of the composition.

45. The method of claim 43, wherein the concentration of the functional substance is within the range of from about 2 to about 15% by weight, based on the total weight of the composition.

46. The method of claim 43, wherein the concentration of the functional substance is within the range of from about 2 to about 6% by weight, based on the total weight of the composition.

47. The method of claim 43, further comprising at least one of a cosmetic, a pharmaceutical, or other topical agents.

48. The method of claim 47, wherein the cosmetic, pharmaceutical and other topical agents comprise one or more ingredient selected from the group consisting of: compounds that improve or eradicate age spots, keratoses and wrinkles; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotionsickness agents; antiinflammatory agents; antihyperkeratolytic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; vitamins; corticosteroids; tanning agents; humectants; hormones; retinoids; gum disease or oral care agents; topical cardiovascular agents; corn, callus and wart removing agents; dipilating agents; and other dermatologicals.

49. The method of claim 47, wherein the cosmetic, pharmaceutical and other topical agents comprise one or more ingredient selected from the group consisting of aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascoryl palimate, atropine, azelaic acid, bacitracin, bemegride, beclomethasone dipropionate, benzophenone, benzoyl peroxide, betamethasone dipropionate, betamethasone valerate, brompheniramine, bupivacaine, butoconazole, calcipotriene, camphor, capsaicin, carbamide peroxide, chitosan, chlorhexidine, chloroxylenol, chlorpheniramine, ciclopirox, clemastine, clindamycin, clioquinol, clobetasol propionate, clotrimazole, coal tar, cromolyn, crotamiton, cycloserine, dehydroepiandrosterone, desoximetasone, dexamethasone, diphenhydramine, doxypin, doxylamine, dyclonine, econazole, erythromycin, estradiol, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, ichthammol, imiquimod, indomethacin, ketoconazole, ketoprofen, kojic acid, lidocaine, meclizine, meclocycline, menthol, mepivacaine, methyl nicotinate, methyl salicylate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocaine, procaine, promethazine propionate, propranolol, pseudoephedrine, pyrethrin, pyrilamine, resorcinol, retinal, 13-cis retinoic acid, retinoic acid, retinol, retinyl acetate, retinyl palmitate, salicylamide, salicylic acid, selenium sulfide, shale tar, sulconazole, sulfur, sulfadiazine, tazarotene, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, vitamin E acetate, wood tar, zinc pyrithione, and mixtures thereof.

50. The method of claim 33, wherein the cosmetic or dermatological disorder is selected from the group consisting of disturbed keratinization, inflammation, defective syntheses of dermal components, and changes associated with intrinsic and extrinsic aging of skin, nail and hair.

51. The method of claim 50, wherein the cosmetic or dermatological disorder is selected from the group consisting of: dryness or looseness of skin, nail and hair; xerosis; ichthyosis; palmar and plantar hyperkeratoses; uneven and rough surface of skin, nail and hair; dandruff; Darier's disease; lichen simplex chronicus; keratoses; acne; pseudofolliculitis barbae; dermatoses; eczema; psoriasis; pruritus; warts; herpes; age spots; lentigines; melasmas; blemished skin; hyperkeratoses; hyperpigmented skin; abnormal or diminished syntheses of collagen, glycosaminoglycans, proteoglycans and elastin, and diminished levels of such components in the dermis; stretch marks; skin lines; fine lines; wrinkles; thinning of skin, nail plate and hair; skin thickening due to elastosis of photoaging, loss or reduction of skin, nail and hair resiliency, elasticity and recoilability; lack of skin, nail and hair lubricants and luster; dull and older-looking skin, nail and hair; fragility and splitting of nail and hair, and skin lightening.

52. The method of claim 50, wherein the changes associated with intrinsic and extrinsic aging of skin are selected from the group consisting of: progressive thinning of skin; fragile skin; deepening of skin lines and fine lines; wrinkles including fine and coarse wrinkles; lusterless skin surface; coarse and uneven skin; loss of skin elasticity and recoilability; blemished and leathery skin; loss of skin lubricating substances; increased numbers of blotches and mottles; nodules; pre-cancerous lesions; pigmented spots and mottled skin; changes in qualities and quantities of collagen and elastic fibers; solar elastosis; decrease in collagen fibers; diminution in the number and diameter of elastic fibers in the papillary dermis; atrophy of the dermis; stretch marks; reduction in subcutaneous adipose tissue; deposition of abnormal elastic materials in the upper dermis; yellowing skin; telangiectatic skin; and older-looking skin.
